(19) United States
(12) Patent Application Publication
(10) Pub. No.: US 2008/0228135 A1
(43) Pub. Date: Sep. 18, 2008

(54) APPARATUS FOR TREATING A DAMAGED SPINAL DISC

(76) Inventor: Elizabeth Ann Snoderly, Murrells Inlet, SC (US)

Correspondence Address: MCNAIR LAW FIRM, P.A., P.O. BOX 10827, GREENVILLE, SC, 29603-0827, US

(21) Appl. No.: 12/042,888

(22) Filed: Mar. 5, 2008

Related U.S. Application Data
(60) Provisional application No. 60/904,995, filed on Mar. 5, 2007.

(51) Int. Cl. A61M 31/00; A61F 7/12; A61B 18/14; A61B 17/24

(52) U.S. Cl. 604/95.04; 604/113; 604/114; 606/110

(57) ABSTRACT

The advantages of this invention are provided by a surgical tool for repairing a damaged spinal disc of a patient comprising: a catheter having a proximal end and a distal end; a guide wire contained within the catheter so that when the guide wire is pulled, the catheter will flex into a non linear shape allowing it to be steered once inserted in the patient; a heating element carried by the catheter, probe or heating member, and connected to a power source so that the heating element can be activated and provide heat to treat a disk; a grasping probe that extends from the catheter so that the grasping probe can be extended out of the catheter; and, a grasping member that can from the distal end of the catheter.

### CLAIM OF PRIORITY

This patent application claims priority on provisional patent application Ser. No. 60/904,995 filed Mar. 5, 2007 entitled Apparatus For Treating A Damaged Spinal Disk

### FIELD OF THE INVENTION

The present invention is directed to an apparatus for treating a ruptured, torn, or bulging disc. More specifically, this invention is an apparatus for performing both Percutaneous Microdiscectomy Decompression (PMD) and Intradiscal Electrothermal Annuloplasty (IDET) without the need for multiple insertions of surgical tools into a patient.

### BACKGROUND OF THE INVENTION

Low back pain occurs in about 90% of all people at some time in their lives. One problem in understanding “low back pain” is that it describes a symptom, not a specific disease. It is becoming clear that chronic low back and leg pain is often the result of damage to spinal discs.

The present invention is directed to a medical treatment tool for the treatment of discs in the lumbar, cervical, or thoracic spine that have suffered from a bulge, rupture, or a tear. The disc in the spine absorbs mechanical loads placed on the spine while also allowing the spine to flex. The disc can be damaged due to a number of reasons including aging, degeneration of the disc, or injury to the spine. A herniated disc can be caused by sufficient pressure on the disc by the vertebrae adjacent to it. This could be caused by lifting a heavy object improperly, by sudden twisting, by the weakening of the disc that can occur with age or other reasons, or by traumatic injury to the back.

When the soft interior of the disc bulges out of its normal location, it places pressure on the spinal cord or nerve root. Therefore, pain, nerve damage, or other damage or adverse symptoms can occur. Damage to the disc can result in tears in the outer shell of the disc. The jelly-like material inside the disc, the disc nucleus, may be forced out through the tears or cracks in the outer shell causing a bulge or break.

Damage to the disc can cause pain and it is desirable to treat this pain. Treatment can include strengthening the annulus of the weakened disc wall and to deaden the painful nerves. One option used to treat patients with certain disc pain is Intradiscal Electrothermal Therapy. This involves passing a flexible catheter through a needle into the painful disc. Wires inside the catheter then heat the disc wall resulting in fibrosis which strengthens as well as desensitizes the disc. This requires the catheter to be inserted into the patient through percutaneous approach.

Once inserted, the wire that is inserted into the disc rests beside the area to be heated and therefore treated. Placement of the wire in the disc can be a difficult problem, particularly when the tear in the disc is larger. When the catheter is inserted into the disc, it is desirable to have it travel around the inner circumference of the outer shell. It is undesirable for the catheter to be caught or hung on the tear and even may protrude through the tear. Further, the catheter is difficult to steer. Therefore, it is advantageous to have a catheter which allows a heating wire to be steerable internal to a disc so that the catheter can travel within the disc and steered around the inner part of the disc around to the inner part of the annulus.

Another adverse effect a damaged disc can have is when the disc material within the disc expands to the point that excess pressure is exerted. In the case of a bulging disc, there can be a tear as well as increased pressure in the disc space. The tear can allow for the pressure in the disc to be relieved. When the tear is repaired, there is no place for the pressure to be relieved and the beneficial results of the tear repair may be undermined. In order to relieve the pressure from excess disc material or Percutaneous Microdiscectomy Decompression can be performed to remove material prior to the disc repair. This increases the chances of achieving good results. In the PMD process, the physician removes disc material through a small cannula using a special tool. This tool rotates rapidly and winds disc material around the tool which is then removed along with the wound disc material. Traditionally, this procedure requires the insertion of the special tool into the disc. To perform PMD and IDET together, two surgical tools would have to be inserted into the patient.

With each insertion of a surgical tool, risks associated with the procedure are increased. Therefore, it would be advantageous to provide for a surgical tool that includes the ability to perform PMD and IDET without having to insert two surgical tools or which only require a single tool insertion.

### SUMMARY OF THE INVENTION

The advantages of this invention are provided by a surgical tool for repairing a damaged spinal disc of a patient comprising: a catheter having a proximal end and a distal end; a guide wire contained within the catheter having a first end extending outward from the proximal end of the catheter and a second end carried by the distal end of the catheter so that when the first end is pulled, the catheter will flex into a non linear shape allowing it to be steered once inserted in the patient; a heating element carried by the distal end of the catheter and connected to a power source so that the heating element can be activated and provide heat at the distal end of the catheter so that damage to a disc can be treated with heat when the catheter is inserted in the patient; a grasping probe contained within the catheter wherein the grasping probe extends outward from the proximal end of the catheter so that a distal end of the grasping probe can be pulled into or extended out of the distal end of the catheter; and, a grasping member carried by the distal end of the grasping probe so that when the grasping probe is extended from the distal end of the catheter, the grasping member grabs disc material contained with the spinal disc so that that the disc material can be removed from the spinal disc when the grasping probe is removed from the patient. The heating member can be carried by the catheter, the probe or a separate heating probe.

### DESCRIPTION OF THE DRAWINGS

The invention will be more readily understood by referring to the following drawings and specification.

FIG. 1 is a cross section of a disc showing the invention inserted in the disc;

FIG. 2 is a cross section of a disc showing the invention inserted in the disc;

FIG. 3 is a cross section of a disc showing the invention inserted in the disc;

FIG. 4 is a side view of the invention;

FIG. 5 is a side view of the invention;

FIG. 6 is a perspective view of the invention;

FIG. 7 is a perspective view of the invention;

FIG. 7A is a perspective view of a portion of the invention;

FIG. 7B is a perspective view of a portion of the invention;

FIG. 8 is a perspective view of the invention;

FIG. 9 is a perspective view of the invention;

FIG. 9A is a prospective view of a portion of the invention; and,

FIG. 9B is a perspective view of a portion of the invention.

### DESCRIPTION OF THE INVENTION

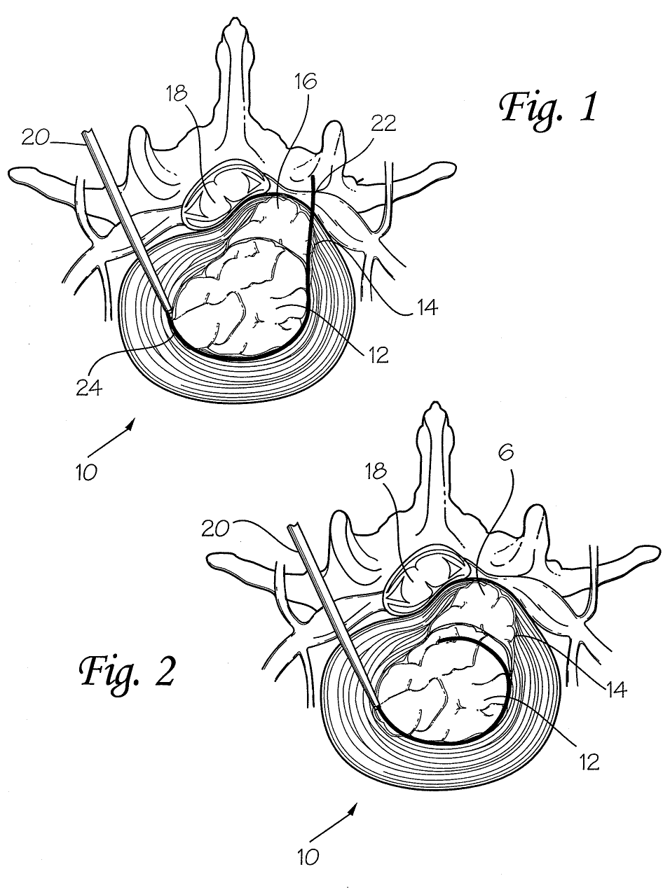

Referring now to FIG. 1, a disc 10 is shown having internal disc material 12. In FIG. 1, a tear 14 has allowed disc material to bulge into area 16. Due to the displacement of the disc material, pressure is placed on spinal cord 18 which can result in pain to the individual. When performing IDET to repair the tear, a surgical tool 20 having a catheter 22 is inserted into the disc at location 24. The tool is inserted in an area away from the tear so as not to worsen the tear. When the tool is inserted at location 24 the catheter portion travels around the circumference of the disc until it reaches the area of tear 14. Once the catheter reaches this area, a heating section near the distal end of the catheter can be actuated to apply heat to the tear to promote the hearing of the tear.

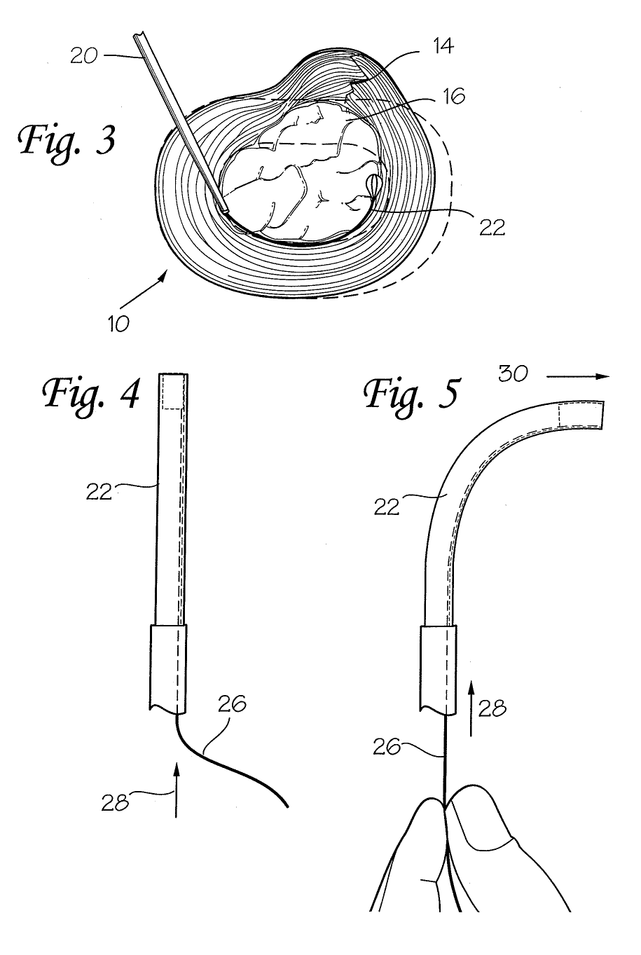

When the catheter travels along the circumference of the disc, it is important that the catheter not extend through the tear as shown in FIG. 1, but to continue to travel around the disc as shown in FIG. 2. Therefore, a guide wire 26 is attached internally to catheter 22 shown in FIG. 3. Catheter 22 is flexible so that when the guide wire is pushed in direction shown as 28, catheter 22 flexes so that the tip of the catheter moves in a direction shown as 30 in FIG. 5. When guide wire 26 is pulled, the catheter can return to more of a linear configuration.

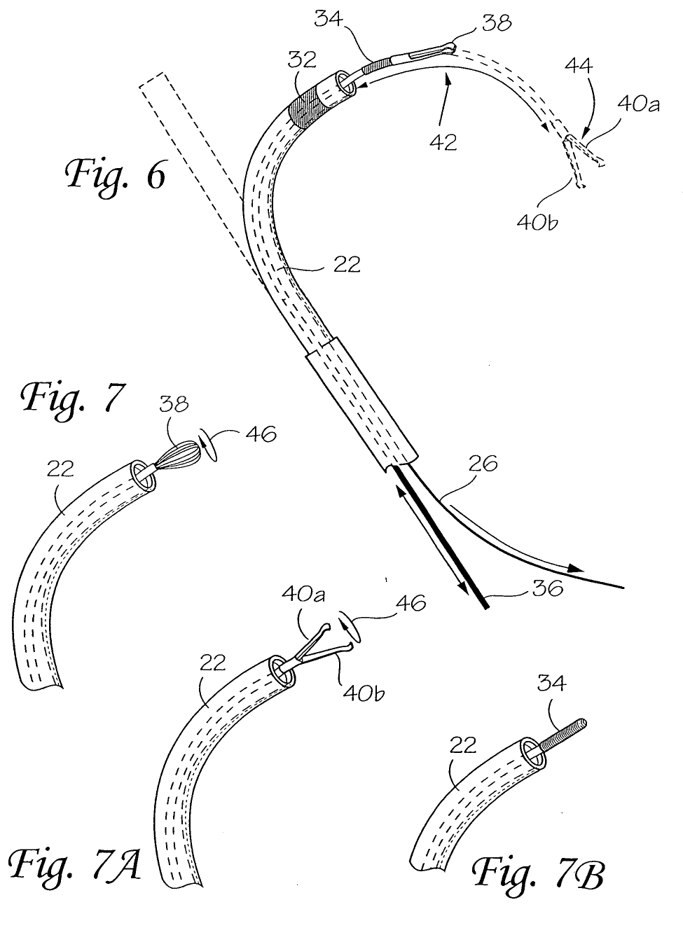

When the catheter is placed within the disc and in the tear location, the tear can be repaired by use of heat therapy as in IDET. In one embodiment, shown in FIG. 6, catheter 22 has an integrated heating element 32. The catheter is directed within the disc so that the heating element is placed adjacent to the tear. When the heating element is actuated, the heat from the probe can cause the material around the tear to “heal”, therefore assisting in repairing the tear. When the guide wire is used to steer the catheter around the circumference of the disc, the catheter can be prevented from entering the tear. Therefore, there is less risk of damage to the already weaken disc due to the insertion of the catheter.

It should also be noted that tool 20 can be inserted directed in to the annular space and it too can be hollow in one embodiment. In this embodiment, probe 22 can be steerable as described herein and can pass through tool 20 and be steered to the desired location within the disc. This allows for a single probe having a heating member and grasping member or separate probes having a heating member and grasping member respectively which are also steerable, to be inserted into the patient without the need to remove tool 20.

Probe 22 can also be hollow allowing for other surgical tools to pass through catheter 22. For example, probe 36 can be inserted into surgical tool 20 and extend out of catheter 22. The probe can have a heating element 34 which can be used to perform IDET. This allows the catheter to be positioned into place and the probe to be inserted after the catheter is properly placed within the disc.

The probe can also have a grasping member 38. In performing PMD, the disc material can be removed using the grasping member to remove disc material from the disc so that pressure is relieved and the tear can be repaired. Otherwise, the expanded disc material can cause another tear or impede the repair of the existing tear. Therefore, the probe can be inserted into the catheter and therefore into the disc, the grasping member can grasp disc material for removal. Then, the heating element can be actuated to repair the tear without having to insert multiple surgical tools into the patient.

A grasping probe carrying a grasping member 38 can have a pair of arms 40a and 40b that have a closed position shown as 42 and an open position shown as 44. In the closed positions, the grasping member can travel through the catheter. Once the grasping member clears the end of catheter 22, the arms can be placed in the open position to grasp disc material and closed to hold onto the disc material. The probe can then be rotated in a direction shown as 46 in FIG. 7A to wind the disc material around the probe so that the disc material can then be pulled through the catheter 22 and therefore removed from the disc. On one embodiment, grasping member 38 can be in the form of a whisk to grasp disc material as shown in FIG. 7. FIG. 7B shows another embodiment of grasping member having grasping arms 40a and 40b that, when used to grasp disc material, can rotate in a direction shown as 46 to wind disc material around the grasping member so that it can be removed from the damaged disc.

It should be noted that the grasping member can be removed from the patient through catheter or hollow member 22 without catheter or hollow member 22 being removed from the patient. Therefore, heating member 35 can be inserted into catheter or hollow member 22 and heating portion 34 used to treat the damaged disc. Therefore, the grasping member and heating member need not be incorporated in one probe, but can be separate members for insertion into catheter.

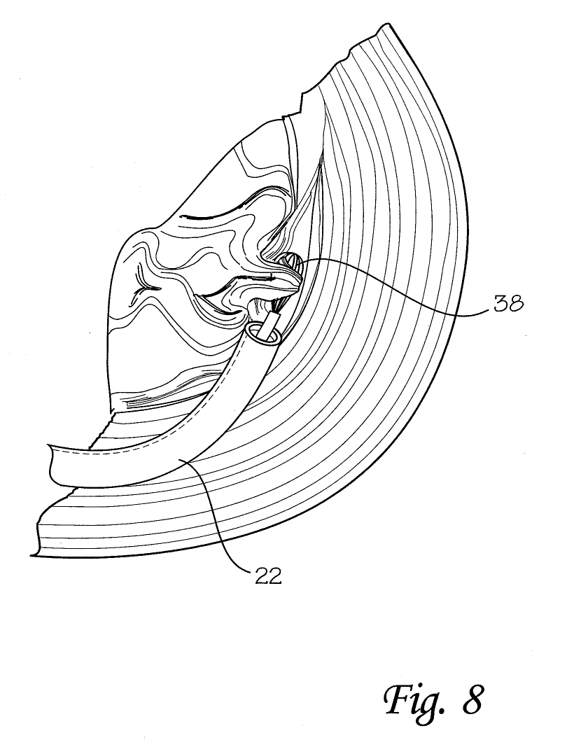

Referring to FIG. 8, once the whisk is inserted into the disc, the grasping member can be rotated and therefore grasp disc material and pull the disc material from the disc through catheter 22.

In one use of the invention, the catheter is inserted into the patient and the disc. The distal end of the catheter is steered in proximity to the tear or location of the disc to be repaired. The probe is extended outward from the distal end of the catheter and the heating element is actuated to assist in repairing the damaged disc. The probe can also be sued to grasp disc material with the grasping member and rotating the probe winds disc material around the grasping member and the probe. The catheter can then be removed from the patient and the disc material within. Further, the probe can be retracted within the catheter prior to removing the catheter.

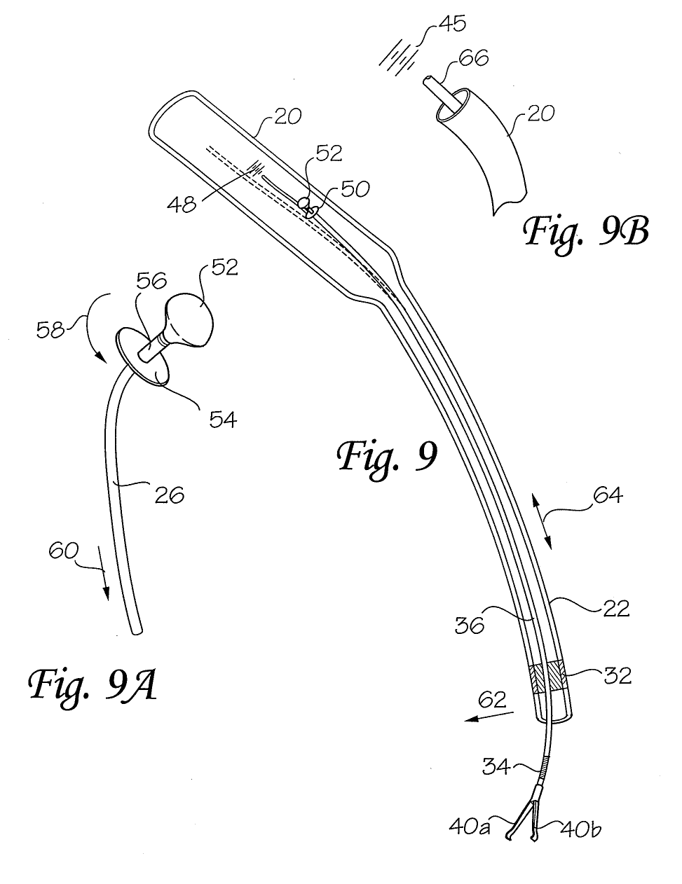

Referring now to FIG. 9, another embodiment is described. Surgical tool 20 is shown having probe guide wire contained within the surgical tool and carried by the probe. The probe guide wire can be carried by guide wire actuating member 52 which can cause the probe guide wire to travel within probe 22. When member 52 (FIG. 9A) is rotated in a direction shown as 58, a carrier plat 54, connected to knob 52 through post 56, causes probe guide wire to move in a direction shown as 60. When probe guide wire moves in direction 60, the distal end of the probe moves in a direction shown as 62 allowing the probe to be steered. Therefore, the probe can be steered once inserted in the patient when the distal end of the probe is extending out of the catheter.

Probe 36 can also be connected to a probe actuating member 50. Probe actuating member 50 can cause probe 36 to travel the directions shown as 64 thereby allowing the distal end of probe 36 to extend beyond the distal end of probe 36 and retract within the probe. Power source 48 (FIG. 9B) can be contained within surgical tool 20 or can be located outside of the surgical tool and connected to the surgical tool by connection 66. In one embodiment, the structure of the probe actuating member can be used in conjunction with the guide wire of the catheter so that an actuating member can be used to actuate the guide wire and therefore flex the catheter so that the catheter can also be steered.

While the invention has been described using specific terms, such description is for illustrative purposes only and it is to be understood that changes and variations may be made without departing from the spirit or scope of the following claims.

What is claimed is:

1. A surgical tool for repairing a damaged spinal disc of a patient comprising:
a catheter having a proximal end and a distal end;
a guide wire contained within said catheter having a first end extending outward from said proximal end of said catheter and a second end carried by said distal end of said catheter so that when said first end is pulled, said catheter will flex into a non linear shape allowing it to be steered once inserted in the patient;
a heating element carried by said distal end of said catheter and connected to a power source so that said heating element can be activated and provide heat at said distal end of said catheter so that damage to a disc can be treated with heat when said catheter is inserted in the patient;
a grasping probe contained within said catheter wherein said grasping probe extends outward from said proximal end of said catheter so that a distal end of said grasping probe can be pulled into or extended out of said distal end of said catheter; and,
a grasping member carried by said distal end of said grasping probe so that when said grasping probe is extended from said distal end of said catheter, said grasping member grabs disc material contained with the spinal disc so that that the disc material can be removed from the spinal disc when said grasping probe is removed from the patient.

2. The apparatus of claim 1 wherein said grasping member includes a first arm and a second arm having an open and a closed position so that said arms will grab disc material when actuated from said open to said closed position.

3. The apparatus of claim 1 wherein said grasping member includes a whisk so that when said grasping member is rotated, said whisk will grab disc material.

4. The apparatus of claim 1 including:
a handle carried by said proximal end of said catheter; and,
a power source carried by said handle and connected to said heating element to provide power to said heating element so that heat can be applied to a damaged disc.

5. The apparatus of claim 1 including:
a handle carried by said proximate end of said catheter; and,
a guide wire actuating member carried by said handle and attached to said first end of said guide wire so that when said guide wire actuating member is actuated, said guide wire is repositioned within said catheter causing said catheter to flex so that said catheter can be steered while being inserted into the patient.

6. The apparatus of claim 1 including:
a handle carried by said proximate end of said catheter; and,
a grasping actuating member carried by said proximal end of said grasping lead wire so that when said grasping actuating member is actuated, said grasping lead wire is inserted into the patient.

7. The apparatus of claim 1 including a probe guide wire contained within said grasping probe having a distal end and connected to said distal end of said grasping probe so that when said probe guide wire is actuated, said grasping probe can be steered.

8. The apparatus of claim 7 including:
a handle carried by said proximate end of said catheter; and,
a grasping actuating member carried by said proximal end of said probe guide wire so that when said grasping actuating member is actuated, said probe guide wire is inserted into the patient and can be steered.

9. A surgical tool for repairing a damaged spinal disc of a patient comprising:
a catheter having a proximal end and a distal end;
a heating element for insertion into said proximal end of said catheter for extending out of said distal end of said catheter and connected to a power source so that said heating element can be activated and provide heat at said distal end of said catheter so that damage to a disc can be treated with heat when said catheter is inserted in the patient;
a grasping probe for being inserted into said proximal end of said catheter for extending out of said distal end of said catheter; and,
a grasping member carried by said distal end of said grasping probe so that when said grasping probe is extended from said distal end of said catheter, said grasping member grabs disc material contained with the spinal disc so that disc material can be removed from the spinal disc when said grasping probe is removed from the patient.

10. The apparatus of claim 9 wherein said grasping member includes a first arm and a second arm having an open and a closed position so that said arms will grab disc material when actuated from said open to said closed position.

11. The apparatus of claim 9 wherein said grasping member includes a whisk so that when said grasping member is rotated, said whisk will grab disc material.

12. The apparatus of claim 9 including a probe guide wire contained within said grasping probe having a distal end and connected to said distal end of said grasping probe so that when said probe guide wire is actuated, said grasping probe can be steered.

13. The apparatus of claim 9 including a probe guide wire contained within said heating element having a distal end and connected to a distal end of said heating element so that when said probe guide wire is actuated, said heating element can be steered.

14. A surgical tool for repairing a damaged spinal disc of a patient comprising:
a catheter having a proximal end and a distal end;
a probe for being inserted into said proximal end of said catheter for extending out of said distal end of said catheter;
a grasping member carried by said distal end of said probe so that when said probe is extended from said distal end of said catheter, said grasping member is rotated to capture disc material contained with a damaged disc that can be removed from the damaged disc when said probe is removed from the patient;
a heating element carried by said distal end of said grasping member and connected to a power source so that said heating element can be activated and provide heat at said distal end of said grasping member so that disc material ca be removed and damage to a disc can be treated with heat when said grasping member is inserted into the spinal disc of the patient.

15. The apparatus of claim 14 wherein said grasping member includes a first arm and a second arm having an open and a closed position so that said arms will grab disc material when actuated from said open to said closed position.

16. The apparatus of claim 14 wherein said grasping member includes a whisk so that when said grasping member is rotated, said whisk will grab disc material.

17. The apparatus of claim 14 including:
a handle carried by said proximal end of said catheter; and,
a power source carried by said handle and connected to said heating element to provide power to said heating element so that heat can be applied to a damaged disc.

18. The apparatus of claim 14 including a guide wire contained within said catheter having a first end extending outward from said proximal end of said catheter and a second end carried by said distal end of said catheter so that when said first end is pulled, said catheter will flex into a non linear shape allowing it to be steered once inserted into a patient.

19. The apparatus of claim 14 including a probe guide wire contained within said probe having a first end extending outward from said proximal end of said probe and a second end carried by said distal end of said probe so that when said first end is pulled, said probe will flex into a non linear shape allowing it to be steered once inserted into a patient.
